(19) United States
(12) Patent Application Publication
(10) Pub. No.: US 2016/0157483 A1
(43) Pub. Date: Jun. 9, 2016

(54) COMPOSITION FOR THE CLEANING AND PROTECTION OF TECHNICAL SURFACES

(71) Applicant: aimecs GmbH, Pfarrkirchen (DE)

(72) Inventors: Hans-Peter Wendel, Balingen (DE); Juergen Weindl, Pfarrkirchen (DE)

(21) Appl. No.: 15/045,094

(22) Filed: Feb. 16, 2016

Related U.S. Application Data
(63) Continuation of application No. PCT/EP2014/067335, filed on Aug. 13, 2014.

(30) Foreign Application Priority Data
Aug. 16, 2013 (DE) 10 2013 108 870.8

(51) Int. Cl. A01N 31/08; A61L 31/10; A61L 31/06; A61L 31/16

(52) U.S. Cl. CPC A01N 31/08; A61L 31/16; A61L 31/10; A61L 31/06; A61L 2300/404

(57) ABSTRACT

A composition for cleaning, disinfecting, protective treatment, or a combination thereof of technical surfaces contains microparticles loaded with an antibacterially effective ingredient. The antibacterially effective ingredient can be totarol. Microparticles loaded with totarol can be used for the coating of medical implants. Medical implants coated with a composition for cleaning, disinfecting, protective treatment or a combination thereof are provided.

### CROSS-REFERENCES TO RELATED APPLICATIONS

This application is a continuation of copending international patent application PCT/EP 2014/067335 filed on Aug. 13, 2014 and designating the U.S., which has been published in German as WO 2015/022366 A1 on Feb. 19, 2015, and claims priority from German patent application DE 10 2013 108 870.8 filed on Aug. 16, 2013. The entire contents of these prior applications are incorporated herein by reference.

### FIELD

The present invention relates to a composition for the cleaning, disinfecting and/or protecting treatment of technical surfaces, comprising at least one antibacterially effective ingredient.

### BACKGROUND

Composition of such kind are well known from everyday life. They contain antibacterially effective ingredients such as alcohols, tensides, etc.

The known compositions are used in industry, household, and hospitals after the first cleaning of technical surfaces, i.e. the removal of dirt particles and other impurities, in order to also clear them from microorganisms and disease germs and, where appropriate, to protect them for a certain period from a renewed contamination with microorganisms or other germs.

In the context of the present invention a “technical surface” refers to a non-biological surface, which requires a regular or one-time disinfection. Such technical surfaces include for example, without limitation, the surfaces of tables, door handles, chairs, trays, beds, technical devices, household appliances, telephones, windows, toilets, office equipment, bathrooms, kitchens, operating rooms, operating devices, and operating gloves.

The company Neroform AG offers, for example, the disinfectant Neroform G for the cleaning of surfaces in the office, hospital and household, which should eliminate 98% of all bacteria within 60 seconds and should almost entirely prevent the new formation of bacterial colonies for over one month via a long-term effect. Neroform G is supposed to protect from infectious diseases, bacteria and fungi.

Neroform G is available as disinfection spray, soaked cloths and liquid disinfectant. The ingredient of Neroform G and its formulation are not known to the applicant.

The efficiency of such composition and its fields of application as well as its long-term effects quite often leave much to be desired. In addition, many microorganisms develop certain resistances in the course of time against the applied disinfectants so that a continuous need for new and improved means of such kind as mentioned at the outset does exist.

### SUMMARY

For this reason, the problem underlying the present invention is to create a new composition of such kind as mentioned at the outset.

This problem is solved by the invention by a composition of such kind as mentioned at the outset, which comprises microparticles loaded with the antibacterially effective ingredient, wherein the microparticles are preferably biodegradable microcapsules or microspherules.

According to the invention “microparticles” refer to mostly spherical solid particles having a size range of between 1 and 1000 μm. As a rule, they are based on biodegradable, biocompatible polymers such as PLA (poly (lactic acid)) or PLGA (poly(lacticco-glycolic acid)). Such microparticles belong to the parenteral extended release drug formulations which have already been established in the market place.

It is known from the US 2010/0003300 A1 to load such microparticles with therapeutic, prophylactic or diagnostic, bioactive reagents, for example with antibiotic or antimicrobial agents, and to inject the compositions produced in such a way into a patient via needles having a small diameter. The composition may also contain a pharmaceutically acceptable carrier, additives and/or solvents.

The composition should effect a controlled and, where applicable, delayed release of the reagents in the body of the patient.

The composition is also intended as a coating for medical devices and implants, for example heart valves and vascular protheses.

Microparticles are characterized by a number of advantages, such as an improved bioavailability, a reduction of systemic side effects as well as an improvement of the compliance and the comfort of the patients.

More and more scientific groups show an increased interest in the inclusion of hydrophobic or hydrophilic active agents and biologicals for a long-term treatment of chronic and neurodegenerative diseases, such as diabetes mellitus and Morbus Parkinson.

Microparticles are produced by various methods. In most cases the method is adapted to the respective active agent and its physical, chemical properties. The production method decides which of the two kinds of microparticles will result. It is distinguished between microcapsules consisting of a core and shell material, and microspheres/spherules consisting of a polymer matrix.

The wall material of the microcapsules encloses the solid, liquid or gaseous core material, respectively, which contains the active agent. For the preparation of microcapsules in the pharmaceutical field mostly the coacervation method, the emulsion method with solvent evaporation/extraction, and the spray drying are used.

Microspherules are microparticles with a diameter of smaller than about 250 μm, in the polymer matrix of which active agents are embedded as homogenously as possible. They are especially suited for a release of the active agent over a period of several weeks. Microspherules result from an emulsion method where the solvent within which the polymer is dissolved, is relatively rapidly vaporized or extracted.

For the production of the composition according to the invention numerous emulsion methods are available. The traditional emulsions are the oil-in-water emulsions (O/W emulsions) and the water-in-oil emulsions (W/O emulsion). The first mentioned emulsion is particularly suited to include hydrophobic active agents into microparticles, thereby achieving inclusion efficiencies of more than 90%, while dissolving or dispersing the active agent in the organic phase.

With the W/O emulsion such results are neither obtainable for hydrophilic nor hydrophobic active agents. Because hydrophilic agents do not or only poorly dissolve in the organic phase, the simple W/O emulsion is completed by an outer phase. As a result, two wide-spread kinds of multiple emulsions are developed, on the one hand a water-in-oil-in-water emulsion (in short: W/O/W) and on the other hand the water-in-oil-in-oil emulsion (in short: W/O/O).

The inventors have realized that, against expectations, for the composition mentioned at the outset intended for the cleaning, disinfection, and protection of technical surfaces outside of the body, such microparticles can be used as carrier for the ingredients.

In the context of the invention “cleaning, disinfection, and protection” also includes the decontamination of technical surfaces or devices.

The loading of the microparticles with the ingredients also results in a continuous release of the active agent over a period of weeks also outside of the body, so that even after several weeks a sufficient disinfection effect is ensured.

Therefore, the release of the ingredients is not effected promptly at the beginning of the application followed by decreasing rapidity, or as an “s-curve”, thus at the beginning and at the end of the effective period with high rapidity and in between with low rapidity. The release kinetics is rather uniform or constant over the effectiveness period, although it is also possible to release a larger amount in a so-called burst at the beginning of the effectiveness period, which then changes to a continuous release.

The ingredients could be various antibacterially effective compounds and its mixtures.

The problem underlying the invention is completely solved in this way.

The present invention further relates to the use of the composition according to the invention for the cleaning, for the disinfection, for the decontamination and/or for the protection of technical surfaces.

In another embodiment of the invention the microparticles are made of a biodegradable and biocompatible polyester, preferably of PLGA.

PLGA has found a wide-spread use in medicine as a self-absorbing suture material or in pharmacy as a depot medicine (drug implant). This polymer consists of defined parts of poly lactic acid (lactide) and polyglycolic acid. The ratio of such acids and the molecular weight of the entire polymer determine the subsequent properties of the implant.

The high proportion of lactide provides for a slower degradation of the implant, but at the same time also for a very slow and low release of the active agent, which makes it difficult to achieve the minimum effective dose of the active agent. However, a higher or more balanced glycolic acid proportion results in a faster degradation and release of the active agent, whereby a desired effect can be reached. There are many different PLGA polymers which strongly differ in their molecular weights and the lactide-glycolide ratio and, as a result, in the application.

For a release of the ingredients within a time period of 6-15 weeks it has been found by the inventors, that a polymer having a lactide-glycolide ratio of 50:50 and a molecular weight of smaller than 30.000 g/mole is particularly suited.

For the production of the microparticles which are loaded according to the invention the Resomer® RG 502 H has proven particularly suited, which is sold by the company Evonik.

After the production the microparticles are available in a dry state.

In another embodiment of the invention the composition contains a solvent for the microparticles loaded with the ingredient, wherein preferably the solvent is selected from the group consisting of hexane, acetone, ethanol, acetonitrile, chloroform, DSMO (dimethyl sulfoxide), methanol, ethyl acetate, and the tenside Brij®30.

Once the new composition is applied from the storage container to the surface which may be effected by wiping using a soaked cloth, spraying or by a direct application of the liquid a kind of a film of the loaded microparticles is formed through the drying, which progressively releases the ingredients via hydrolytic processes, which then can develop their antimicrobial effect.

In an embodiment of the invention the new composition can also be present in a soaked cloth, in particular a cleaning cloth, wound dressing, cover drape, a soaked face protection mask or soaked surgical clothes.

In a further embodiment of the invention the ingredient at least comprises a totarol compound, preferably synthetically produced totarol or totarol obtained from a natural source.

Totarol is a tricyclic aromatic diterpene which, as ((+)-totarol, comprises antimicrobial and antioxidative properties. Since many years it is proposed for a use in cosmetic and pharmaceutical products. The Cosmetic, Toilettry, and Fragrance Association has allocated to totarol the CFTA reference number 7277.

Due to its strongly effective antioxidative and antibacterial properties totarol extracts are already used as additives in toothpastes and cosmetics.

Totarol can be obtained as natural substance from various plant materials, in particular from the heartwood of a New Zealand podocarpus species (Podocarpus totara).

The IUPAC name of totarol is: (4bS,8aS)-4b,8,8-Trimethyl-1-propan-2-yl-5,6,7,8a,9,10-hexahydrophenanthren-2-ol. The CAS number is 511-15-9.

Totarol has the chemical structure as shown in FIG. 9.

The WO 2005/073154 A1 describes appropriate methods for the production of totarol containing extracts from appropriate plant material. Such extracts should be further processed into solvent-free products which are effective against gram-positive and gram-negative bacteria and may be used as cleaning and disinfection means for industry and household, cosmetics, pharmaceuticals, skin care and personal care products as well as for dental care.

If formulated as orally applicable medicament the products should be provided inter alia in the form of capsules, tablets, pastilles, sirupe, mouth washes, toothpastes, chewing gums, and mouth sprays.

If they should be used as topically applicable formulation the products should be provided inter alia as lotion, cream, gel, spray, cleaning liquid, shampoo, powder, hydrogel or wound dressing.

The WO 2005/073154 A1 describes appropriate methods for the production of extracts, however no methods for the production of the products.

Because the natural resources of totarol are limited the EP 2 143 703 B1 relates to a method for the chemical synthesis of (+)-totarol.

It is known from the EP 1 925 301 A1 that totarol and its antimicrobially effective esters as well as their diastereomeres can be used for the production of pharmaceutical products and nutraceuticals and for the treatment of inflammatory diseases such as arthritis.

The synthesis of antibacterially effective totarol derivatives are described in Evans et. al. “The synthesis and Antibacterial Activity of Totarol Derivatives”, Part 1 in Bioorganics & Medicinal Chemistry 7 (1999), 1953-1964; Part 2 in Bioorganics & Medicinal Chemistry 8 (2000), 1653-1662; Part 3 in Bioorganics & Medicinal Chemistry 8 (2000), 1663-1975.

According to that, totarol is also effective against multi-resistant Staphylococcus aureus (MRSA).

The authors report about experiments which show that the minimum concentration where totarol still has an antibacterial effect in vitro is 2 μg/ml. The authors also demonstrate that totarol is toxic in a concentration of more than 5 μg/ml and inhibits the growth of human cells. They propose further experiments in order to examine whether the range between these concentrations provide a sufficient safety window so that totarol can be used for the treatment of human bacterial diseases.

The U.S. Pat. No. 6,881,756 B2 describes the use of totarol and its pharmaceutically effective esters for the topical treatment of pain and itching. To this end, totarol is formulated with a topical carrier, examples of which are indicated as solutions, emulsions, gels, micells and liposomes.

The U.S. Pat. No. 6,881,756 B2 does not describe methods for the production of the formulations.

In the context of the present invention “totarol” means a composition of the structure as shown in FIG. 9, and “totarol derivatives” means a composition derived from totarol, which is antimicrobially effective, within the meaning of the publications cited above, in particular its derivatives and esters as well as the corresponding diastereomeres.

A totarol compound that is used according to the invention means an ingredient which includes totarol, totarol derivatives and/or its mixtures.

The inventors have realized that totarol compounds can be loaded onto microparticles in a simple and effective manner without interfering with the antibacterial effect.

They were able to show in first experiments that microencapsulated totarol has antibacterial effects against Streptococcus gordonii even after a longer incubation period. The effect of encapsulated totarol was less than such of free totarol, which was caused by the slower release of totarol from the microcapsules.

Because of the antimicrobial effect of totarol compounds as described above the inventors provide a novel composition of the above-mentioned kind, where both the ingredient as well as its provision in form of loaded microparticles differ from the prior art.

The delayed release of the totarol compound from the microparticles loaded therewith now allows on the one side the continuous release of totarol both in vitro as well as in vivo, so that the antibacterial effect can be maintained over a longer period of time. Because the totarol is stored in the microparticles in addition a large amount of totarol can be held in stock, without the totarol exerting its toxic effect to human cells. This allows the release of totarol in vivo over longer periods of time.

When using Resomer 502® RG 502 H microcapsules could be produced with a weight ratio of totarol and Resomer of between 70 and 77%, the diameters of which were 60 to 140 μm.

Further disinfecting compounds and its mixtures can be provided as further ingredients, in order to achieve a wide-spread effect of the new means as large as possible against various microorganisms and pathogenic agents.

The further disinfecting compounds can be stored in separate microparticles so that the composition according to the invention contains first microparticles loaded with totarol, and second microparticles loaded with another compound.

In doing so, the advantage is achieved that the composition may comprise at least two different active substances which cannot be stored in contact to each other over a longer period of time, however which are loaded into separate particles so that they are separated from each other.

Against the background of the above development and the experiences resulting therefrom the inventors have further realized that the microparticles loaded with a totarol compound as used in the composition according to the invention can also be used as a coating for the surfaces of medical devices and implants, preferably of vascular prostheses, further preferably of external sides of vascular prostheses, and finally, however not limited thereto, for thoraco-abdominal, iliacal and popliteal stents and stent grafts, peripheral stents, for further common vascular prostheses such as PTFE, Dacron, PU, absorbable polymer and saccharide stents, each in dilatable, self-expandable, structured or covered stent form.

Therefore, the invention also is a medical implant having a surface on which a coating is provided, which comprises microparticles according to the above description, which are loaded with a totarol compound.

The invention further is a medical implant which at least partially consists of absorbable material, preferably of saccharide compounds, wherein the implant contains microparticles loaded with at least one antibacterially effective ingredient, preferably it contains the above-described microparticles, and wherein the microparticles are incorporated into the absorbable material, wherein further preferably the new composition is incorporated into the absorbable material.

By incorporating or mixing the microparticles into the bioabsorbable implant structure, for example into the support structure of a vascular prosthesis, the ingredients are gradually released in the cause of the entire degradation time period of the implant, thereby conferring a long protection period.

The microparticles can be immobilized on the surfaces of the implants for example via adhesion promoters such as highly viscous PLGA, PVA or with a biological glue, for example fibrin glue. Experiments of the inventors show for example that vascular prostheses can be provided with a coating of totarol loaded microparticles in a reliable and stable manner, if a 2% solution of polyvinyl alcohol (PVA) is used as an adhesion promoter.

The invention further is a coating material comprising at least first microparticles loaded with at least one antibacterially effective ingredient according to the above description.

This coating material can be produced and stored in a dry state up to its use or can be purchased from an external supplier.

According to the invention the ingredient includes at least one totarol compound, preferably synthetically produced totarol or totarol from a natural source.

The inventors have realized for the first time that totarol and its derivatives can be inter alia used for the treatment of existing pathological vascular diseases and for preventing prostheses infections, especially if the introduced vascular prosthesis comprises a coating or a biodegradable structure with a totarol content. The loaded microparticles result in a prolonged release kinetics over several weeks which contribute to the prevention of prostheses infections in a long-acting manner.

Hasse B, Husmann L, Zinkernagel A, Weber R, Lachat M, Mayer D. Vascular graft infections. Swiss Med Wkly. 2013 Jan. 24; 143, report that prostheses infections are difficult to be treated. For this reason, according to the invention it is of great advantage that because of the coating of the prosthesis according to the invention no adhesion of bacteria is taking place, thus a consecutive biofilm formation can be avoided.

Keidar Z, Engel A, Hoffman A, Israel O, Nitecki S. J Nucl Med. 2007 August; 48(8):1230-6. Prosthetic vascular graft infection: the role of 18F-FDG PET/CT, report on a relatively late point in time of the occurrence of a graft infection so that the combatting of a graft infection should not be the objective but the prevention of the formation because once a biofilm has been generated it is nearly impossible to treat it therapeutically and it can only be tackled aggressively by means of surgery.

In the implants coated according to the invention the occurrence of a graft infection is reliably prevented even over longer periods of time after the implantation because of the delayed release of totarol compound.

As already mentioned totarol is provided with a long-lasting high toxicity. For this reason, without an additional release mechanism as developed by the inventors in the human medical field the use of the totarol compound according to the invention would not be possible over a longer period of time. Because after a surgical procedure infections can occur in a timely delayed manner a long-lasting release in order to combat bacterial strains is very advantageous.

Experiments of the inventors also show that totarol and totarol-loaded microparticles do not have negative influences on the hemostatic system, so that the uses of totarol or totarol-loaded microparticles according to the invention do not meet with objections.

In an embodiment of the invention the medical implants can be pace-makers and its electrodes, artificial hips, bone substitutes, marrow nails, patches, meshes, self-expanding and balloon-expanding, bioabsorbable and non-bioabsorbable stents and covered stents, endografts, urostents, brachial stents etc., the surfaces of which being in contact with tissue are coated according to the invention.

It is also possible to provide mecial devices, including instruments and for example catheters or balloons, with a coating which contains totarol-loaded microparticles to treat or prevent infections in case of a contact with tissue.

Preferably the ingredient may additionally contain a further pharmacological compound, preferably cytostatic or cytotoxic drugs, such as Rapamycin, Paclitaxel, further antibiotics (e.g. Minocyclin-Rifampicin), silver, nanosilver, sulfonamines, antimicrobial peptides (AMPs).

In an embodiment of the invention the implant is a vascular prosthesis, a stent graft or a vascular support, where the new composition is provided on the respective outer surface as a coating.

Vascular prostheses are implants which are introduced into the body to replace damaged sections of natural blood vessels. Vascular prostheses comprise plastic hoses which are attached to the stumps of blood vessels.

When the artificial blood vessels are supported from the inside by metallic structures it is referred to stent grafts.

Vascular supports in the meaning of the present invention are intravascular implants which are also referred to as stents. Such stents are radially expandable endoprostheses which are transluminally implanted into vessels, for example blood vessels, oesophagus, intestinal tract etc., and which are then radially expanded so that they attach to the inside of the wall of the vessel.

Stents are for example used to treat or strengthen blood vessels in aneurysms, lesions, stenoses and/or to prevent a restenosis in the vascular system. They can be self-expanding or can be actively expanded by a radial force which is exercised from the interior, e.g. when they are mounted onto a balloon.

A “radially expanding vascular support” in the context of the present invention both refers to self expanding but also to actively expandable vascular supports.

The vascular supports comprise a hollow cylindrical body with a wall of braces or branches connected with each other, which may establish a radially permeable and elastic structure. It is also known to use non-permeable (covered) stents. The stents can be self-expanding or balloon-expandable.

The outer diameter of the body in the expanded state corresponds to the inner diameter of the vessel to be supported, at the wall of which the vascular support is attached while exercising a radial force caused by its flexible and elastic structure. In the longitudinal direction the body of the vascular support is open for the passage of media or substances transported in the supported vessel.

The vascular supports can have a branched configuration and/or comprise lateral openings to receive or to enable the access to the branching vessels.

Especially for stents it is further known to coat their surfaces luminally or abluminally with active substances, especially with drugs or to provide drug reservoirs in the structure of the stents or to use microporous braces or branches to temporarily store the drug. The drug is then locally dispersed to the vessel wall in order to prevent e.g. restinoses because of proliferation of the surrounding tissue.

As a consequence, by means of correspondingly coated stents or stents provided with reservoirs the active substances can be, so to say, in situ administered to the surrounding tissue in a targeted manner. The coating of stents, i.e. of vascular prostheses, with active substances is also desirable because the biocompatibility of the implants is improved by means of which e.g. the development of thromboses in surfaces being contact with blood can be prevented.

Such a supply of the vascular wall with drugs is especially important for stents with a pure metal surface, so-called bare-metal stents (BMS) because such stents show a relatively high rate of restenosis of approximately 30%. The reason for this is the vessel injury caused by the implantation which results in a proliferation of the smooth muscle cells.

To prevent or attenuate a proliferation of the smooth muscle cells the stents are coated with cytostatic or cytotoxic drugs, such as Rapamycin or Paclitaxel. They are then called drug-eluting stents (DES).

For this, the drugs are bound to the surface of the stent via a polymeric, biologically degradable binder or introduced into a polymer matrix on the surface of the stent. Subsequent to the implantation of the stents after the degradation of the polymer the drug enters the vascular wall.

As a bioabsorbable binder essentially several polyesters on the basis of lactic and/or glycolic acid are used, which degrade in the body without residuals. A use is known of PLA, PLLA and PLGA with different monomer ratios. Such polymeres in parts significantly differ from each other in their mechanical and chemical properties.

The WO 2013/007589 A1 describes a stent where the binder is a oligo(D,L-lactate-co-glycolate), which comprises a molecular weight (Mₗ) which is about 3,000 Dalton, wherein the monomere ratio of lactic acid and glycolic acid in the oligomere is about 1:1.

The binder is applied as “closed coating” onto the outer surface of the stent which covers at least the entire abluminal surface of the vascular support, and which does not chip-off again in larger areas after crimping or a later dilating of the vascular support. As an active substance Rapamicin with the binder was inserted into the closed coating in a weight ratio of 1:1.

The oligomer used as a binder in the WO 2013/007589 A1 is commercially available from the company Evonik under the trade name Resomer® Condensate 50:50 Mₙ 2300. A polymer, namely PLGA with a molecular weight of about 10.000 Dalton has been examined by way of comparison, which is distributed by Evonic as Resomer® RG 502 H.

In the WO 2013/007589 A1 it is shown that after contacting with tissue only a coating containing the oligomer disintegrates after six week by more than 99%.

From the EP 1 977 772 A2 a stent is known having a coating of PLGA on its surface.

The inventors of the present application have now realized that especially PLGA is particularly suited for the production of a coating of microparticles loaded with a totarol compound, to achieve a constant release of the ingredient over a time period of several weeks.

Starting from the WO 2013/007589 A1 this was not to be expected.

In case of the use of a stent or a vascular prosthesis having a coating using the totarol compounds as an ingredient prostheses infections are effectively prevented.

In the context of the present invention “prostheses infections” refer to infectious inflammations which have a pathological origin or develop after the implantation on surfaces of implants, namely via microorganisms introduced by the implant surface or during surgery or via pathogens already present in the patient. According to the invention these microorganisms are eliminated by the gradual release of the totarol and/or the totarol derivatives from the microparticles over a time period of several weeks.

The new composition can comprise first microparticles loaded with totarol compounds and second microparticles loaded with a further ingredient, for example an active substance such as Rapamicin. As a result the antibacterial effect of totarol is combined with the antiproliferative effect of cytostatically or cytotoxically effective substances, respectively.

According to the invention it is further intended that on the inventive coating or directly on the surface of the implants an outer layer is provided which contains a totarol compound, preferably synthetically produced totarol or totarol from a natural source, wherein further preferably the outer layer is produced by emerging of the implant which may comprise the coating into a solution which contains a totarol compound, preferably synthetically produced totarol or totarol from a natural source.

This has the advantage that the coating which contains microparticles loaded with a totarol compound according to the invention, is provided within outer layer of non-capsulated totarol, which seals the coating and after the implantation emits the totarol rapidly in a desired amount and in a first release (a so-called burst).

In particular application cases it may be sufficient to refrain from a coating with the microparticles and to provide the totarol-containing outer layer, preferably via a immersion coating, directly on the surface of the implant. Thereby, by the surface structure of the implant a delayed release of the totarol compound can be effected.

In general, the present invention also relates to the use of totarol compound for the coating of surfaces of medical devices and implants.

Further advantages result from the description and the enclosed drawing.

It goes without saying that the before-mentioned features and those to be explained in the following can be used not only in the combinations as indicated in each case, but also in other combinations or in isolated position without departing the scope of the present invention.

### BRIEF DESCRIPTION OF THE DRAWINGS

Embodiments of the invention are shown in the enclosed drawing and are explained in further details in the following description.

FIG. 1 a schematic, and cutted side view of a stent before its delatation which is introduced into a vessel and provided with the coating according to the invention;

FIG. 2 a schematic, cutted side view of a vascular prothesis with a coating according to the invention and an outer layer;

FIG. 3 a bar diagram presenting first measured values;

FIG. 4 a curve showing the release kinetics of totarol from microparticles over a time period of 37 days;

FIG. 5 the comparison of measurements of the optical density (OD) at 600 nm for suspensions of S. aureus which were treated with 0.1 mg/ml totarol (totarol), with EtAc, or which remain untreated (untreated);

FIG. 6 bar charts showing the effect on the hemostatic system of totarol and microparticles loaded with totarol;

FIG. 7 further bar charts showing the effect on the hemostatic system of totarol and microparticles loaded with totarol;

FIG. 8 a bar chart for the viability of HEK-cells; and

FIG. 9 the chemical formula of totarol.

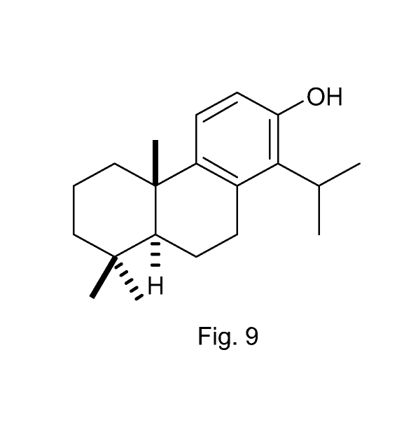

### DETAILED DESCRIPTION

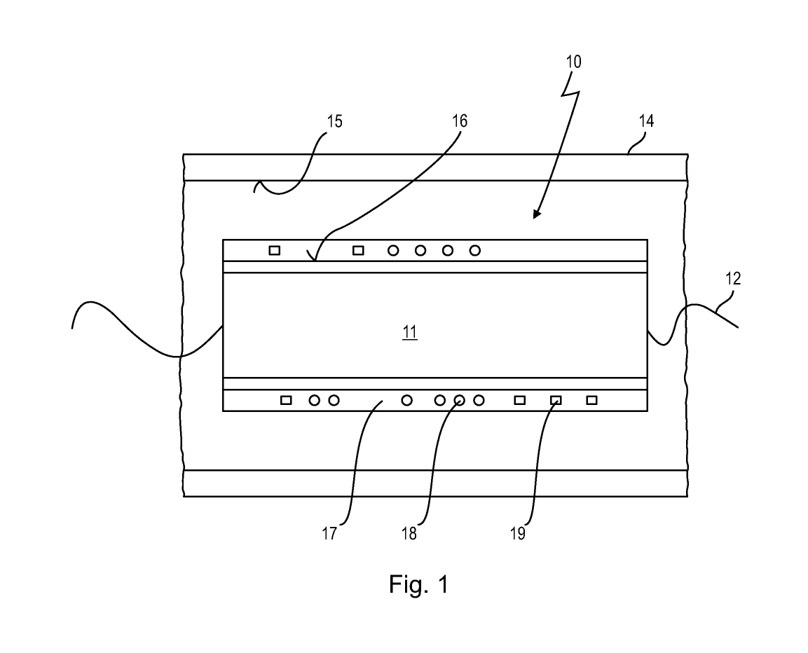

In FIG. 1 it is shown a schematic and non-scaled side view of a stent 10, which is crimped onto a balloon 11. The balloon 11 is introduced via a guide wire 12 into a vessel 14, after the expanding by the balloon 11 it comes into contact with the vessel's wall 15.

The stent 10 comprises an outer surface 16 onto which a coating was applied which is schematically indicated at 17. The coating contains first and second microparticles identified of 18 and 19.

The first microparticles 18 were loaded with totarol in a manner as described in the following. The second microparticles 19 were loaded with Rapamycin.

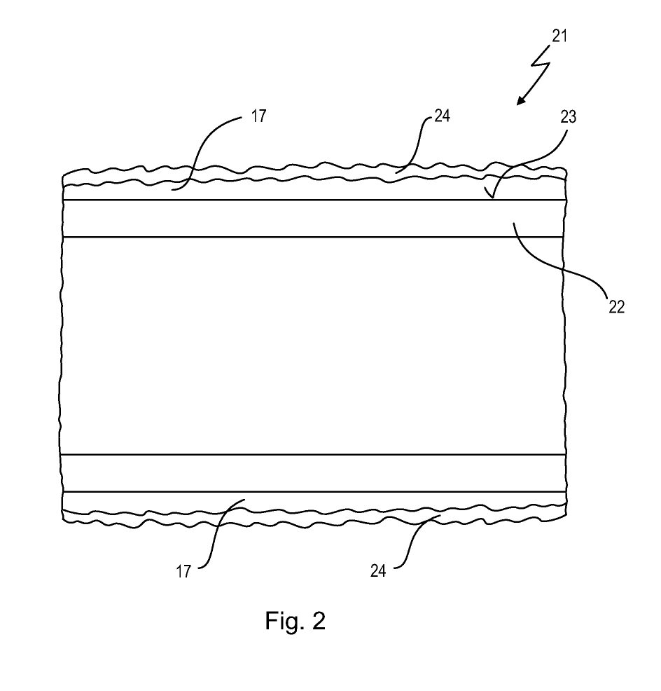

FIG. 2 shows a schematic and non-scaled side view of a vascular prosthesis 21 with a merely indicated, here cylindrical wall 22, onto the surface 23 of which the coating 17 known from FIG. 1 is applied, onto which an outer layer 24 of a totarol compound is applied via immersing coating. In particular applications for this vascular prosthesis 21 it can be refrained from the coating 17 so that the outer layer 24 is directly positioned on the surface 23.

The immersion coating was tested for ePTFE vascular prostheses having a length of 3 cm and an inner diameter of 6 mm. For this, in a Petri dish 600 mg of totarol was dissolved in 5 ml of ethyl acetate to produce an almost saturated totarol solution. The vascular prostheses were immersed for 5 minutes into this solution so that they were able to soak with totarol solution, followed by 30 min of drying. By taking the difference of the weight of the vascular prostheses before and after the immersing and drying the amount of the absorbed totarol was determined as 30 mg per vascular prosthesis.

The microparticles 18 loaded with totarol were produced as described in the following examples I and II.

### EXAMPLES

### Example I

### Production of W/O/W Loaded Totarol Microparticles, Evaporation Method

First step: Production of a continuous (CP) and an organic phase (OP), both phases are produced on a magnetic stirrer.

The CP consists of destilled water, sodium chloride (NaCl), the emulgators Tween®20 and polyvinyl alcohol (PVA), and sodium hydroxide (NaOH).

At first, the NaCl is dissolved in destilled water with stirring (300 rpm). Then the PVA is added and heated to 80° C. with further stirring (450 rpm). This temperature is maintained as long as the PVA has completely dissolved. Then the CP is cooled down to 40° C. with further stirring (600 rpm). After this temperature is reached the Tween®20 is added and cooled down to room temperature with further stirring (500 rpm).

The OP which is produced in parallel to the CP consists of the active agent totarol, the polymer Resomer® 502, ethyl acetate and the tenside Brij®30.

Firstly, the active agent totarol and the Resomer®502 is pre-deposited and dissolved in ethyl acetate with stirring (230 rpm). After about 5 min all substances should be complete dissolved. With further stirring (230 rpm) the Brij®30 is added and stirred for further 5 min.

Then in a first step both phases are emulsified into each other in a ratio of 1:1. For this, the CP is added to the OP drop by drop (1 ml per minute). This step is taking place in a beaker glass with permanent stirring (280 rpm). In this process the preemulsion (W/O) is formed which is stirred for 20 min at the same rotational speed.

In the next step again a part of the CP is added to pre-emulsion, exactly 7-times the used solvent in the OP. By this the multiple emulsion W/O/W is formed. After the addition it is further stirred (300 rpm) for one hour at room temperature. During this time the microparticles start to harden.

In the last step again one part of the CP is added, namely 19-times the used solvent in the OP. It is further stirred (300 rpm) for one hour, wherein the temperature is increased to 24° C.

In the following steps the microparticles are sifted out and washed three times in destilled water. In the following they are air-dried for 24 hours.

### Example II

### Production of O/W Loaded Microparticles, Evaporation Method

First step: Production of a continuous (CP) and an organic phase (OP), both phases are produced on a magnetic stirrer.

The CP consists of destilled water, sodium chloride (NaCl), the emulgators Tween®20 and polyvinyl alcohol (PVA).

At first, the NaCl is dissolved in destilled water with stirring (300 rpm). Then the PVA is added and heated to 80° C. with further stirring (450 rpm). This temperature is maintained as long as the PVA is completely dissolved. Then the CP is cooled to 40° C. with further stirring (600 rpm). As soon as this temperature is reached the Tween®20 is added and cooled to room temperature with further stirring (500 rpm). OP: This phase is produced in parallel with the CP.

The OP consists of the active agent totarol, the polymer Resomer® 502, ethyl acetate and the tenside Brij®30.

At first, the active agent totarol and the Resomer®502 is predeposited and dissolved in ethyl acetate with stirring (230 rpm). After approximately 5 min all substances should be completely dissolved. With further stirring (230 rpm) the Brij®30 is added and stirred for further 5 min.

40-times the solvent used in the OP is predeposited as CP and the OP is completely added to the CP without stirring. Then it is stirred at 1100 rpm for 1.5 min. After this time the rotational speed is reduced to 350 rpm and it is stirred for 3.5 hours at 25° C.

In the following steps the microparticles are sieved and washed with destilled water for three times. Then they are air-dried for 24 hours.

The weight proportion of the totarol at the microparticles was, depending on the batch, between 70 and 77%, the diameters of the microparticles were about 60 to 140 μm.

The microparticles loaded with totarol were then tested for their antibacterial effect against Streptococcus gordonii (S.G.). For this purpose, 5 mg of totarol-loaded microparticles were given into 6 well plates and covered with a solution of S.G.

For comparison, 5 mg of unloaded microparticles, 5 mg of free totarol, pure medium (Schaedler Medium of the company BD), medium with S.G. and medium with S.G. and 4% P/S (penicillin streptomycin) as positive control were measured.

By means of the change of the optical density the growth of S.G. was determined. The starting density of S.G. was 1.8.

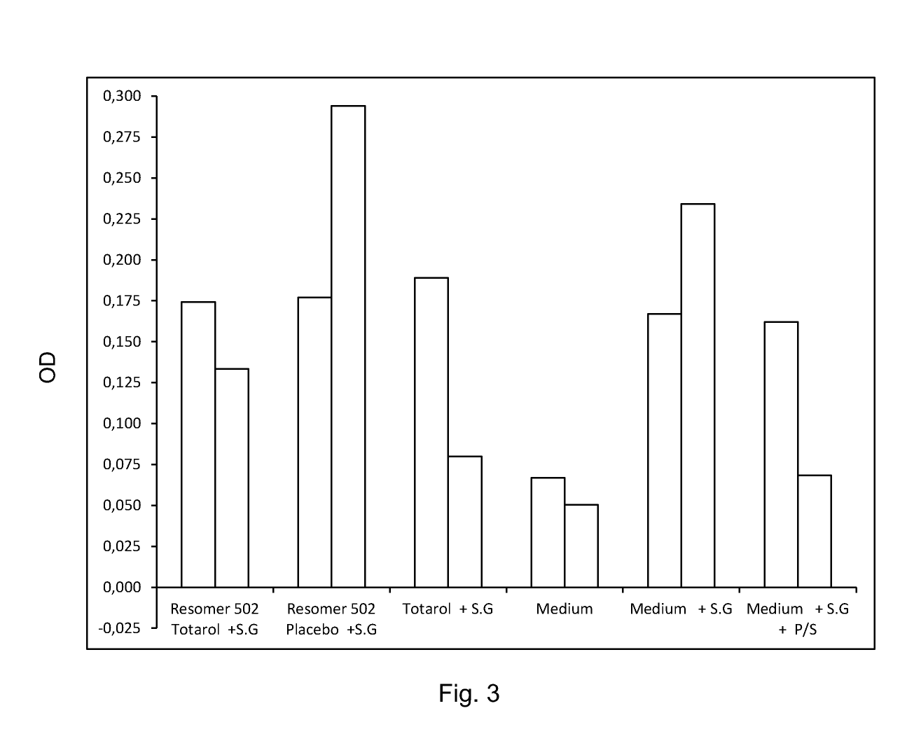

In FIG. 3 the measured results at zero hour (left columns) and after 24 hours (right columns) is shown.

It can be seen that S.G. were grown in the samples with unloaded microparticles and pure medium, whereas free totarol and P/S result in a stronger reduction of the bacterial concentration than totarol-loaded microparticles. This demonstrates that the encapsulated totarol is active, however works slower than free totarol.

### Example III

### Antibacterial Effect of Microparticles Loaded with Totarol

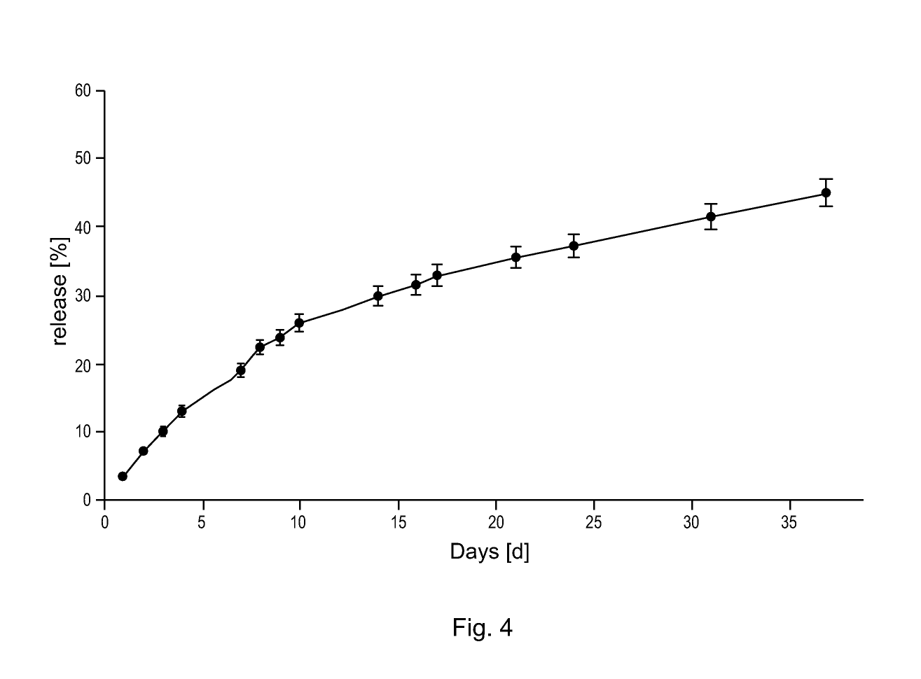

The mean particle size of the microparticles produced by the above-described method and loaded with totarol in one experiment was 156 μm. The amount of encapsulated totarol was 90% of the used totarol. The release kinetics of the totarol is shown in FIG. 4. Accordingly, the release took place continuously over a time period of at least 37 days by 50%.

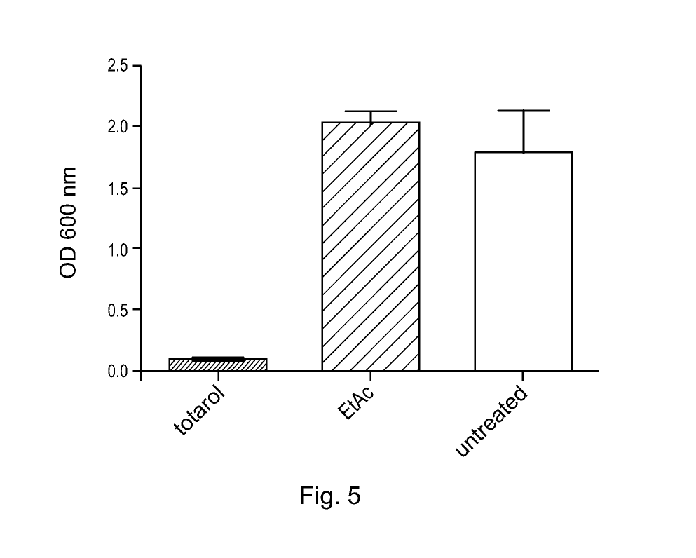

The antibacterial effect on Staphylococcus aureus (S. aureus) of totarol and the microparticles loaded with totarol was tested in vitro in a further experiment. It was shown that a concentration of 0.1 mg/ml of totarol dissolved in ethyl acetate (EtAc) was sufficient to inhibit the growth of S. aureus in suspension after 6 hours of incubation. FIG. 5 shows the comparison of measurements of the optical density (OD) at 600 nm for S. aureus suspensions, which were treated with 0.1 mg/ml totarol (totarol), with EtAc, or which were not treated (untreated).

In further experiments the S. aureus bacteria were seeded onto agar plates and treated with 1 mg of pure totarol, 10 mg of unloaded microparticles, and 10 mg of microparticles loaded with about 1 mg of totarol. After 24 hours of incubation at 37° C. both the culture with pure totarol as well as the culture with the loaded microparticles show a clear inhibition zone.

The experiment was repeated with filter paper which was soaked with pure totarol, with microparticles loaded with totarol and with EtAc, and which was incubated on agar plates, onto which S. aureus was cultivated. After 24 hours of incubation at 37° C., in comparison with the EtAc control, both the culture with pure totarol as well as the culture with the loaded microparticles showed a clear inhibition zone around the filter paper.

### Example IV

### Hemocompatibility of Microparticles Loaded with Totarol

Finally in vitro tests were performed where totarol and microparticles loaded with totarol were brought into contact with whole blood. The effect on erythrozytes, platelets, leucozytes as well as the complement and coagulation cascade was examined. The hemokompatibility was tested with concentrations of totarol and microparticles loaded with totarol, which showed a sufficient antibacterial effect in vitro.

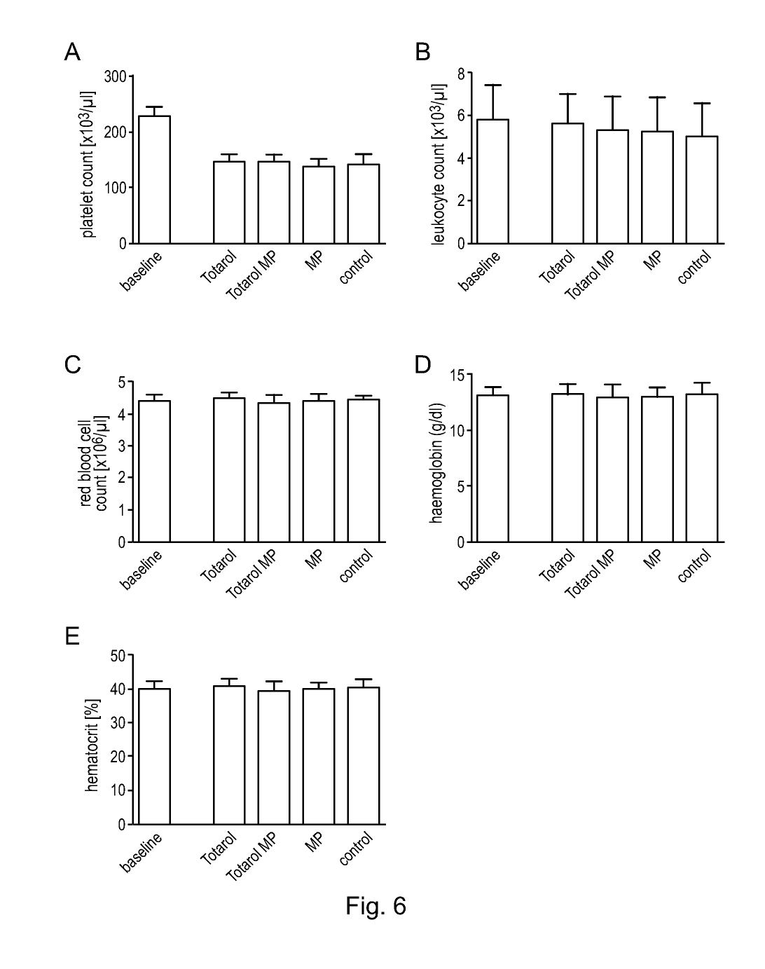

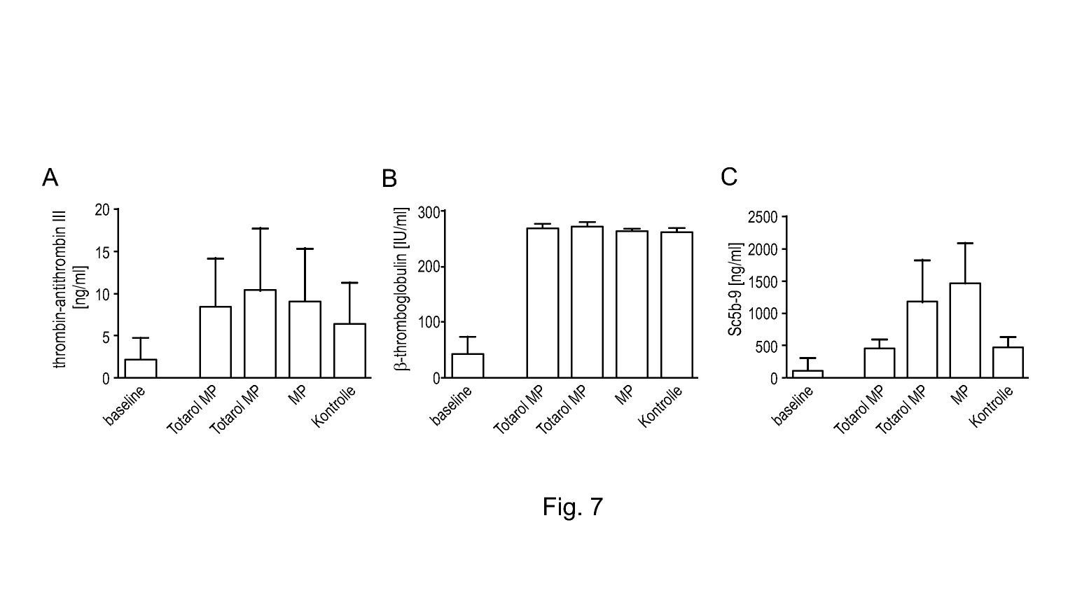

The measurements were made according ISO 10993-4, in determining various markers with a central importance within the hemostatic system. The data show that in comparison with the control group and the treatment with unloaded microparticles neither totarol nor the microparticles loaded with totarol have negative effects on the hemostatic system; see FIG. 6 and FIG. 7

FIG. 6 shows bar charts for the number of platelets, leucocytes, and erythrocytes as well as values for hemoglobin and hematokrit, each measured before (baseline) and after the treatment of fresh human whole blood with 0.1 mg/ml of totarol (totarol), with 1 mg/ml of totarol loaded onto microparticles (totarol MP), with 1 mg/ml of unloaded microparticles (MP) and buffer (control), each incubated for 1 hour. Shown are the means values and standard deviations (n=3).

FIG. 7 shows bar charts for the concentrations of thrombin-antithrombin Ill-, beta-thromboglobulin- and ScSb-9, each measured before (baseline) and after the treatment of fresh human whole blood with 0.1 mg/ml of totarol (totarol), with 1 mg/ml of totarol loaded onto microparticles (totarol MP), with 1 mg/ml of unloaded microparticles (MP) and buffer (control), each incubated for 1 hour. Shown are the mean values and standard deviations (n=3).

All data show a good hemocompatibility both for totarol as well as for the microparticles loaded with totarol. No biologically relevant change during the blood contact could be observed.

### Example V

### Effect of Microparticles Loaded with Totarol on HEK

The cell viability of human embryonic kidney cells (HEK) was analyzed by means of MTT after the incubation with microparticles loaded with totarol (final concentration 1 mg/ml) over a time period of 24 hours. MTT is a test for cell viability using the dye tetrazolium MTT 3-(4,5-dimethylthiazol-2-yl)-2,5-diphenyltetrazolium bromide.

The results show that microparticles loaded with totarol do not have an influence on the viability when they are used in concentrations which are antimicrobially effective.

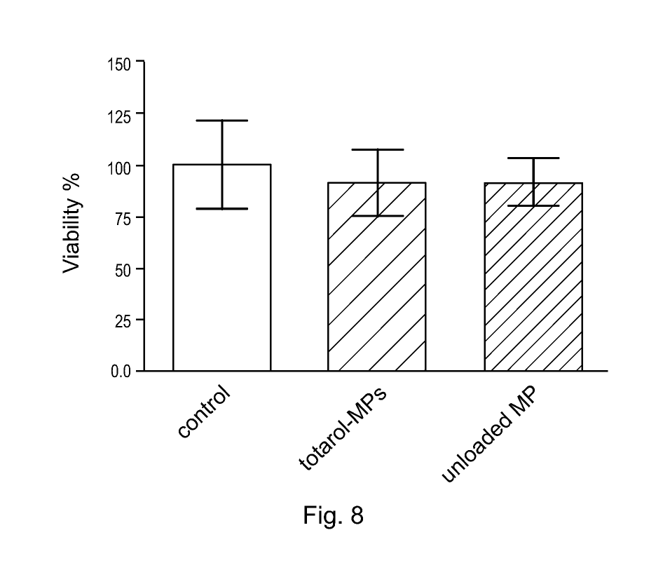

FIG. 8 shows a bar chart for the viability of HEK cells after 24 hours of incubation with 1 mg/ml of totarol loaded onto microparticles (totarol MPs) and with 1 mg/ml of unloaded microparticles (unloaded MPs), in comparison to an untreated control (control). Shown are mean values and standard deviations (n=3).

What is claimed is:

1. A composition for a cleaning, disinfecting, protective treatment of a technical surface, wherein the composition comprises at least one antibacterially effective ingredient, wherein the composition comprises microparticles loaded with the at least one antibacterially effective ingredient.

2. The composition of claim 1, wherein the microparticles are biodegradable microcapsules or microspherules.

3. The composition of claim 2, wherein the microparticles are made of a biodegradable polyester.

4. The composition of claim 3, wherein the microparticles are made of poly(L-lactide-co-glycolide).

5. The composition of claim 4, wherein the microparticles are made of a poly(L-lactide-co-glycolide) with a lactide-glycolide ratio of 50:50.

6. The composition of claim 4, wherein the microparticles are made of a poly(L-lactide-co-glycolide) with a molecular weight of smaller than 30.000 g/mole.

7. The composition of claim 1, wherein the composition comprises a solvent for the microparticles loaded with the at least one antibacterially effective ingredient.

8. The composition of claim 7, wherein the solvent is selected from the group consisting of hexane, acetone, ethanol, acetonitrile, chloroform, DSMO (dimethyl sulfoxide), methanol, ethyl acetate and the tenside Brij®30.

9. The composition of claim 1, wherein the composition is provided in a form selected from the group consisting of a spray, a soaked cloth, in a cleaning cloth, a wound dressing, a cover cloth, a soaked mouth mask, a soaked surgical clothing and a liquid.

10. The composition of claim 1, wherein the at least one antibacterially effective ingredient comprises at least a totarol compound.

11. The composition of claim 10, wherein the at least one antibacterially effective ingredient is a synthetically produced totarol or totarol from a natural source.

12. The composition of claim 10, wherein the ingredient additionally contains a further disinfecting compound.

13. A coating material comprising the composition of claim 1.

14. A medical implant with a surface on which a coating is provided, wherein the coating comprises the coating material of claim 13.

15. The medical implant of claim 14, wherein the medical implant is selected from the group consisting of a vascular prosthesis, a vascular support and a stent graft.

16. The medical implant of claim 15, wherein the surface is an outer surface of the vascular prosthesis.

17. The medical implant of claim 15, wherein the coating comprises an outer layer, wherein the outer layer comprises a totarol compound.

18. The medical implant of claim 14, wherein instead of the coating on the surface an outer layer is provided, wherein the outer layer comprises a totarol compound.

19. The medical implant of claim 17, wherein the outer layer is produced by immersing the implant into a solution, wherein the solution comprises a totarol compound.

20. The medical implant of claim 14, wherein the microparticles are immobilized on the surface.

21. The medical implant of claim 20, wherein the microparticles are immobilized via an adhesion promoter.

22. A medical implant at least partially comprising an absorbable material, wherein the absorbable material comprises microparticles, wherein the microparticles are loaded with at least one antibacterially effective ingredient, wherein the microparticles are incorporated into the absorbable material.

23. The medical implant of claim 22, wherein a composition for a cleaning, disinfecting, protective treatment of a technical surface, wherein the composition comprises at least one antibacterially effective ingredient, wherein the composition comprises microparticles loaded with the at least one antibacterially effective ingredient, is incorporated into the absorbable material.

24. A medical apparatus having a surface on which a coating is provided, wherein the coating comprises the coating material of claim 13.
